(19) United States
(12) Patent Application Publication
(10) Pub. No.: US 2026/0335612 A1
(43) Pub. Date: Oct. 29, 2026

(54) RADIAL ACCESS CATHETER

(71) Applicant: Otsuka Medical Devices Co., Ltd., Tokyo (JP)

(72) Inventors: James MAZZONE, San Jose, CA (US); Eric DAILEY, San Jose, CA (US); Desmond CHEUNG, San Jose, CA (US)

(21) Appl. No.: 19/110,807

(22) PCT Filed: Sep. 9, 2023
(86) PCT No.: PCT/IB2023/058942

§ 371 Date: Mar. 11, 2025

Related U.S. Application Data
(60) Provisional application No. 63/375,357, filed on Sep. 12, 2022.

(51) Int. Cl. A61M 25/10; A61M 25/00; A61M 39/00; A61M 39/24; A61N 7/00; A61N 7/02

(52) U.S. Cl. CPC A61M 25/10186; A61M 25/0043; A61M 39/24; A61N 7/02; A61M 2039/0009; A61M 2039/242; A61M 2039/2433; A61M 2205/0216; A61M 2205/3331; A61N 2007/0021

(57) ABSTRACT

A catheter includes a catheter shaft deliverable through a radial access approach. The catheter shaft has a fluid lumen. A balloon is mounted on the catheter shaft and has an interior in fluid communication with the fluid lumen. An ultrasound transducer is in the interior. A flow control device has an inlet port to received fluid passing through the interior from the fluid lumen. The flow control device is configured to vent the fluid to a surrounding environment when the fluid has a predetermined pressure. Other embodiments are also described and claimed.

This application claims the benefit of priority of U.S. Provisional Patent Application No. 63/375,357, filed on Sep. 12, 2022, titled “Radial Access Catheter,” which is incorporated herein by reference in its entirety to provide continuity of disclosure.

### BACKGROUND

### Field

This application relates generally to minimally-invasive apparatuses, systems and methods that provide energy delivery to a targeted anatomical location of a subject, and more specifically, to catheter-based, intraluminal apparatuses for the treatment of tissue, such as nerve tissue.

### Background Information

According to the Centers for Disease Control and Prevention (CDC), about one in every three adults suffer from high blood pressure, also known as hypertension. Left untreated, hypertension can result in renal disease, arrhythmias and heart failure. In recent years, the treatment of hypertension has focused on minimally invasive interventional approaches to inactivate the renal nerves surrounding the renal artery. Autonomic nerves tend to follow blood vessels to the organs that they innervate. Catheters may reach specific structure that may be proximate to the lumens in which they travel. For example, one system employs a radio frequency (RF) generator connected to a catheter having multiple electrodes placed against the intima of the renal artery and used to create an electrical field in the vessel wall and surrounding tissue that results in resistive (ohmic) heating of the tissue to a temperature sufficient to ablate the tissue and the renal nerve passing through that tissue. To treat all the renal nerves surrounding the renal arteries, the RF electrodes are repositioned several times around the inside of the renal artery. However, the relatively confined electric fields created by the RF electrodes may miss some of the renal nerves, leading to an incomplete treatment. Additionally, to heat the renal nerves, the RF electrodes must contact the intima, posing a risk of damage or necrosis to the intima, which in turn can lead to thrombus formation, fibrosis of the vessel wall, mechanical weakening of the vessel and possible vessel dissection.

Another approach to renal nerve deactivation is the use of high-intensity focused ultrasound (HIFU), which relies on vibrational energy to cause frictional heating and disruption of the tissue, and in turn, raise the tissue temperature sufficiently to cause ablation or remodeling.

U.S. Pat. Nos. 9,943,666, 9,981,108, and 10,039,901 to Warnking, U.S. Pat. Nos. 9,700,372, 9,707,034, and 10,368,944 to Schaer, and U.S. Pat. Nos. 10,350,440 and 10,456,605 to Taylor, the entire contents of each of which is incorporated by reference herein, disclose a system that uses unfocused ultrasound to ablate nerves. Embodiments of the system include an ultrasound transducer positioned along a distal end of a catheter designed to be inserted into a blood vessel (e.g., the renal artery). Electrical cabling, which is received within a cabling lumen of the catheter, can be used to power the ultrasound transducer. The ultrasound transducer emits one or more therapeutic doses of unfocused ultrasound energy, which heats the tissue adjacent to the body lumen within which the transducer is disposed. The system may also include a balloon mounted at the distal end of the catheter used to circulate cooling fluid both prior to, during, and after activation of the transducer to cool the transducer and help prevent thermal damage to the interior surface of the blood vessel wall while the nerves are being heated and damaged at depth. Circulation of the cooling fluid occurs through two fluid lumens-an input fluid lumen that carries fluid distally to the balloon, and an output fluid lumen that returns fluid proximally from the balloon.

Such a design enables creation of one or more ablation zones sufficient to achieve long-term nerve inactivation at different locations around the circumference of the blood vessel, thereby treating a patient's hypertension while mitigating damage to the blood vessel and surrounding organs.

The ultrasound transducer may include first and second electrodes which are arranged on either side of a cylindrical piezoelectric material, such as lead zirconate titanate (PZT). To energize the transducer, a voltage is applied across the first and the second electrodes at frequencies selected to cause the piezoelectric material to resonate, thereby generating vibration energy that is emitted radially outward from the transducer. The transducer is designed to provide a generally uniform and predictable emission profile.

Systems that use unfocused ultrasound to ablate nerves can be delivered to a target anatomy via various access routes. For example, the catheter carrying the transducer can be inserted via a femoral access route through the femoral artery.

### SUMMARY

The present invention is defined in the independent claims. Further embodiments of the invention are defined in the dependent claims.

A catheter is provided herein. The catheter includes a catheter shaft having a fluid lumen. The catheter includes a balloon mounted on the catheter shaft and having an interior in fluid communication with the fluid lumen. The catheter includes an ultrasound transducer in the interior. The catheter includes a flow control device having an inlet port to receive fluid passing through the interior from the fluid lumen. The flow control device is configured to vent the fluid to a surrounding environment when the fluid has a predetermined pressure.

A catheter is provided herein. The catheter includes a balloon having an interior. The catheter includes an ultrasound transducer in the interior. The catheter includes a catheter shaft including an outer member having a central lumen, an inner member extending through the central lumen and having a fluid lumen to deliver fluid to the interior, and one or more steering wires extending through the central lumen between an inner wall of the outer member and an outer wall of the inner member. The one or more steering wires connect to the outer member or the inner member at an anchor point proximal to the balloon.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The various features of the present disclosure and the manner of attaining them will be described in greater detail with reference to the following description, claims, and drawings, wherein reference numerals are reused, where appropriate, to indicate a correspondence between the referenced items, and wherein:

FIG. 1 illustrates an ultrasound-based tissue treatment system, in accordance with an embodiment.

FIG. 2 illustrates a side view of selected components of the ultrasound-based tissue treatment system introduced in FIG. 1, in accordance with an embodiment.

FIG. 3 illustrates a side view of selected components of the ultrasound-based tissue treatment system introduced in FIG. 1, in accordance with an embodiment.

FIG. 4 illustrates a perspective view of selected components of the ultrasound-based tissue treatment system introduced in FIG. 1, inserted into a body lumen, in accordance with an embodiment.

FIG. 5 illustrates a longitudinal cross-sectional view of a distal portion of a catheter of an ultrasound-based tissue treatment system, in accordance with an embodiment.

FIG. 6 is a sectional view, taken about line A-A of FIG. 5, of the distal portion of catheter of the ultrasound-based tissue treatment system, in accordance with an embodiment.

FIG. 7 is a sectional view, taken about line A-A of FIG. 5, of the distal portion of catheter of the ultrasound-based tissue treatment system, in accordance with an embodiment.

FIG. 8 is a side view of a distal portion of a catheter of an ultrasound-based tissue treatment system having a flow control device, in accordance with an embodiment.

FIG. 9 is a sectional view of a flow control device of a catheter of an ultrasound-based tissue treatment system, in accordance with an embodiment.

FIG. 10 is a sectional view of a flow control device of a catheter of an ultrasound-based tissue treatment system, in accordance with an embodiment.

FIG. 11 is a sectional view of a flow control device of a catheter of an ultrasound-based tissue treatment system, in accordance with an embodiment.

FIG. 12 is a side view of a flow control device of a catheter of an ultrasound-based tissue treatment system, in accordance with an embodiment.

FIG. 13 is a sectional view of a flow control device of a catheter of an ultrasound-based tissue treatment system, in accordance with an embodiment.

FIG. 14 is a sectional view of a flow control device of a catheter of an ultrasound-based tissue treatment system, in accordance with an embodiment.

FIG. 15 is a sectional view of a flow control device of a catheter of an ultrasound-based tissue treatment system, in accordance with an embodiment.

FIG. 16 is a sectional view of a flow control device of a catheter of an ultrasound-based tissue treatment system, in accordance with an embodiment.

FIG. 17 is a sectional view of a flow control device of a catheter of an ultrasound-based tissue treatment system, in accordance with an embodiment.

FIG. 18 is a sectional view of a catheter shaft of a catheter of an ultrasound-based tissue treatment system, in accordance with an embodiment.

FIG. 19 is a side view of a catheter of an ultrasound-based tissue treatment system, in accordance with an embodiment.

FIG. 20 is a side view of a catheter of an ultrasound-based tissue treatment system, in accordance with an embodiment.

FIG. 21 is a side view of a catheter of an ultrasound-based tissue treatment system, in accordance with an embodiment.

FIG. 22 is a side view of a catheter of an ultrasound-based tissue treatment system, in accordance with an embodiment.

FIG. 23 is a schematic view of an ultrasound-based tissue treatment system, in accordance with an embodiment.

### DETAILED DESCRIPTION

Systems that use unfocused ultrasound energy to treat tissue, and methods of using the same are provided herein. In certain embodiments, acoustic-based tissue treatment transducers, apparatuses, systems, and portions thereof, are provided. The systems may be catheter-based. The systems may be delivered intraluminally (e.g., intravascularly) so as to place a transducer within a target anatomical region of the subject, for example, within a suitable body lumen such as a blood vessel. Once properly positioned within the target anatomical region, the transducer can be activated to deliver unfocused ultrasonic energy radially outward so as to suitably heat, and thus treat, tissue within the target anatomical region. The transducer or piezoelectric material can be activated at a frequency, duration, and energy level suitable for treating the targeted tissue. In one non-limiting example, unfocused ultrasonic energy generated by the transducer or piezoelectric material or radio frequency (RF) energy transmitted by the electrodes may target select nerve tissue of the subject, and may heat such tissue in such a manner as to neuromodulate (e.g., fully or partially ablate, necrose, or stimulate) the nerve tissue.

In a manner such as described in the Warnking, Schaer, and Taylor patents mentioned above, neuromodulating renal nerves may be used to treat various conditions, e.g., hypertension, chronic kidney disease, atrial fibrillation, autonomic nervous system for use in treating a variety of medical conditions, arrhythmia, heart failure, end stage renal disease, myocardial infarction, anxiety, contrast nephropathy, diabetes, metabolic disorder and insulin resistance, etc. It should be appreciated, however, that the balloon catheters suitably may be used to treat other nerves and conditions, e.g., sympathetic nerves of the hepatic plexus within a hepatic artery responsible for blood glucose levels important to treating diabetes, or any suitable tissue, e.g., heart tissue triggering an abnormal heart rhythm, and is not limited to use in treating (e.g., neuromodulating) renal nerve tissue. In another example, a tissue treatment catheter is used to ablate sympathetic nerves of the renal arteries and a hepatic artery to treat diabetes or other metabolic disorders. In certain embodiments, the tissue treatment catheters are used to treat an autoimmune and/or inflammatory condition, such as rheumatoid arthritis, sepsis, Crohn's disease, ulcerative colitis, and/or gastrointestinal motility disorders by neuromodulating sympathetic nerves within one or more of a splenic artery, celiac trunk, superior or inferior mesenteric artery. In certain embodiments, the tissue treatment catheter is used to ablate nerve fibers in the celiac ganglion and/or renal arteries to treat hypertension. In certain embodiments, the transducers are used to treat pain, such as pain associated with pancreatic cancer, by, e.g., neuromodulating nerves that innervate the pancreas. Ultrasound or RF energy may also be used to ablate nerves of both the pulmonary vein and the renal arteries to treat atrial fibrillation. In still other examples, ultrasound or RF energy may additionally or alternatively be used to ablate nerves innervating a carotid body in order to treat hypertension and/or chronic kidney disease.

In intraluminal systems, ultrasound transducers may be disposed within balloons that are filled with a cooling fluid before and during treatment. More particularly, a balloon may surround the transducer. The balloon may contact the interior surface (e.g., intima) of the body lumen. In certain embodiments, the transducer may be used to output an acoustic signal when the balloon fully occludes a body lumen, and the cooling fluid within the balloon may be used to cool both the body lumen and the transducer. In certain embodiments, the balloon may surround the transducer in order to cool the transducer during sonications, but the balloon may not contact or occlude the body lumen, and the blood within the body lumen may be relied upon to cool the body lumen instead of the cooling fluid.

In certain embodiments, the transducer may be connected to a controller using one or more electrically conductive wires. The controller may be configured to apply power or deliver electrical signals to the transducer. In an embodiment, the controller generates radiofrequency (RF) signals to the transducer. The balloon ablation treatment apparatus may comprise one or more lumens, e.g., a guidewire lumen, one or more fluid lumens, and/or a cable lumen.

Existing tissue treatment systems are sized to be delivered to a target anatomy via a femoral access route. The femoral access route, however, can be painful to a patient, can require lengthy procedural times, and may lead to health complications. Delivery through a radial access route through a radial artery can reduce pain, procedural times, and complications. More particularly, radial access is a more comfortable route of delivery than through a groin, and is associated with fewer complications, such as bleeding.

In order to provide a catheter suitable for tissue treatment via a radial access route, it is advantageous to provide a longer length catheter than is required for access through the femoral artery. More particularly, radial access can require longer length catheters than is required for femoral access. The increase in length, however, can require an increase in cross-sectional area of the fluid lumens used to circulate cooling fluid to the transducer within the balloon. Increasing the cross-sectional area of the fluid lumens can maintain a pressure and fluid flow rate suitable for safe and effective energy delivery. Furthermore, larger diameter fluid lumens can be primed more reliably, and thus, the increase in cross-sectional areas of the fluid lumen can promote effective priming of the system. On the other hand, the radial artery is typically smaller than the femoral artery, and an overall diameter of the catheter must be decreased to 5 French or smaller for patient comfort and safety. In other words, the countervailing requirements of increased fluid lumen diameter and decreased overall catheter diameter limit the ability of existing catheter configurations to be adapted to radial access platforms.

In an aspect, a catheter having a cross-sectional area and length suitable for radial access is provided. The catheter can include a single fluid lumen having a cross-sectional area suitable for providing a safe and effective nerve ablation and priming of the system. In an aspect, the catheter does not require a second fluid lumen, unlike existing tissue treatment systems, and thus, the catheter is capable of delivery to a target anatomy through a radial access route. The elimination of the second fluid lumen can allow the single fluid lumen to be sized to be large enough to achieve sufficient cooling fluid flow to the transducer and small enough to provide a catheter shaft that can be delivered through the radial access route. The catheter can include a flow control device to pass fluid delivered to the catheter from a fluid reservoir into a surrounding environment. The fluid can therefore flow distally through the catheter to cool a transducer of the catheter, and then vent into the surrounding environment. Venting the fluid, rather than returning the fluid to the fluid reservoir, can eliminate the need for the second fluid lumen and makes the catheter capable of delivery to the target anatomy through the radial access route.

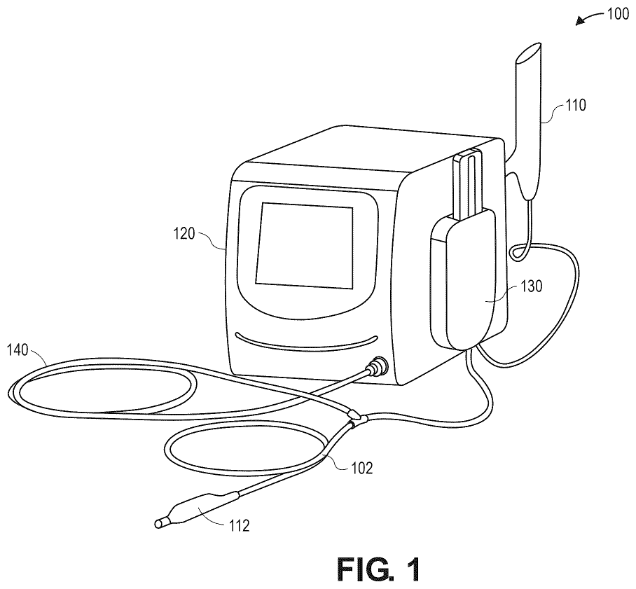

FIGS. 1, 2, and 3 illustrate features of an ultrasound-based tissue treatment system, according to various configurations provided herein. Referring to FIG. 1, an ultrasound-based tissue treatment system is shown in accordance with an embodiment. The tissue treatment system 100 is shown as including a catheter 102, a controller 120, and a connection cable 140. In certain embodiments, the system 100 further includes an ultrasound transducer within a balloon 112, a reservoir 110, a fluid transfer cartridge 130, and a control mechanism, such as a handheld remote control.

In the embodiment shown in FIG. 1, the controller 120 is shown as being connected to the catheter 102 through the cartridge 130 and the connection cable 140. In certain embodiments, the controller 120 interfaces with the cartridge 130 to provide a cooling fluid to the catheter 102 for selectively inflating and deflating the balloon 112. The balloon 112 can be made from, e.g., nylon, a polyimide film, a thermoplastic elastomer (such as those marked under the trademark PEBAX™), a medical-grade thermoplastic polyurethane elastomer (such as Pellethane®, Isothane®, or other suitable polymers or any combination thereof), but is not limited thereto.

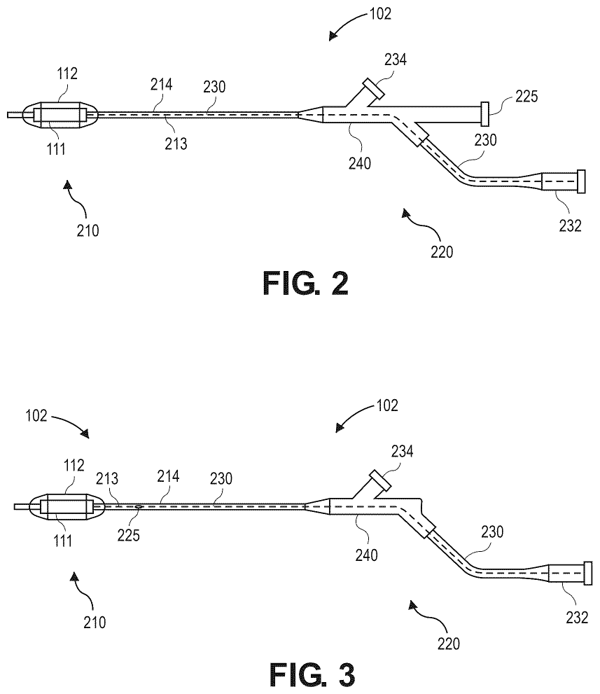

Referring to FIG. 2, a side view of an ultrasound-based tissue treatment system is shown in accordance with an embodiment. The tissue treatment catheter 102 can include a distal region 210 and a proximal region 220. The catheter 102 may have a length that depends on a treatment application. For example, in certain embodiments suitable for, e.g., renal denervation through a radial access delivery method, the catheter 102 can have a working length of 150 to 160 cm, e.g., 155 cm. Furthermore, an overall length of the catheter 102 for such application, including a length of electrical cabling 230 extending to an electrical coupling 232, can be longer. More particularly, the cabling 230 can have a length of about 305 cm from the proximal hub 240 to the electrical coupling 232.

The catheter 102 can have a profile that is suitable to accessing a renal artery through the radial access locations. For example, the catheter 102 may include a catheter shaft 214 having a shaft diameter that is 4 to 6 French in diameter, e.g., less than or equal to 5 French. The profile is facilitated in part by the catheter shaft 214 having an outer diameter in a range of 0.050 to 0.060 inch, e.g., 0.057 inch.

The distal region 210 of the tissue treatment catheter 102 may be a portion of the device that is advanced into a target anatomy, e.g., a target vessel having a vessel wall, to treat the target vessel. The distal region 210 can include the balloon 112 mounted on the catheter shaft 214. The catheter shaft 214 can be an elongated tubular structure that extends longitudinally from a proximal end to a distal end. The balloon 112 can be mounted and supported on the catheter shaft 214 at the distal end. Furthermore, the ultrasound transducer 111 can be mounted on the catheter shaft 214 and contained within an interior of the balloon 112. Accordingly, the catheter shaft 214 can facilitate delivery of a cooling fluid to the balloon 112 and delivery of electrical energy to the transducer 111.

The catheter shaft 214 can include one or more lumens that may be used as fluid conduits, electrical cabling passageways, guidewire lumens, and/or the like. In an embodiment, for example, the catheter shaft 214 can include a guidewire lumen 213 that is shaped, sized and otherwise configured to receive a guidewire. In an embodiment, the guidewire lumen 213 is an over-the-wire type guidewire lumen, extending from a distal tip of the catheter 102 through an entire length of the catheter shaft 214 to an exit port 225 in the proximal hub 240 of the catheter 102. As described below, the lumen(s) of the catheter shaft 214 may also communicate inflation/cooling fluid from the proximal region 220 to the balloon 112 during balloon expansion.

In an embodiment, a transducer 111 is mounted on the catheter shaft 214 at the distal region 210, within an interior of the balloon 112. The transducer 111 can be an ultrasound transducer used to emit energy toward the vessel wall. For example, the transducer 111 can emit ultrasound energy circumferentially, e.g., 360 degrees, around the vessel wall. In an embodiment, electrical cabling 230 extends from the proximal region 220 to the distal region 210, and is connected to the transducer 111 to generate energy for emission to target tissue.

The ultrasound transducer 111 may include first and second electrodes that are arranged on either side of a cylindrical piezoelectric material, such as lead zirconate titanate (PZT). To energize the transducer 111, a voltage is applied across the first and the second electrodes at frequencies selected to cause the piezoelectric material to resonate, thereby generating vibration energy that is emitted radially outward from the transducer 111. The transducer 111 is designed to provide a generally uniform and predictable emission profile, to inhibit damage to surrounding non-target tissue. In addition, a cooling fluid is circulated through the balloon 112, both prior to, during, and after activation of the transducer 111, so as to reduce heating of an inner lining of the body lumen and to cool the transducer 111. In this manner, the peak temperatures achieved by tissue within the cooling zone remain lower than for tissue located outside the cooling zone.

The proximal region 220 may include one or more connectors or couplings. The connectors or couplings can be electrically connected to the transducer 111 via the electrical cabling 230. For example, the proximal region 220 may include one or more electrical coupling 232 that connects to a proximal end of the electrical cabling 230. A distal end of the electrical cabling 230 can be connected to the transducer 111.

The catheter 102 may be coupled to the controller 120 by connecting the electrical coupling 232 to the connection cable 140. The connection cable 140 may be removably connected to the controller 120 and/or the catheter 102 via a port on the controller 120 and/or the catheter 102. Accordingly, the controller 120 can be used with several catheters 102 during a procedure by disconnecting the coupling of a first catheter, exchanging the first catheter with a second catheter, and connecting a coupling of the second catheter to the controller 120. In certain embodiments, e.g., where only one catheter needs to be used during a procedure, the connection cable 140 may be permanently connected to the controller 120.

In certain embodiments, the proximal region 220 of the catheter 102 may further include one or more fluidic ports. For example, the proximal hub 240 can include a fluidic port 234, via which an expandable member, e.g., the balloon 112, may be fluidly coupled to the reservoir 110 (FIG. 1). The reservoir 110 can therefore supply cooling fluid to the balloon 112 through the fluidic port 234. The reservoir 110 optionally may be included with the controller 120, e.g., attached to the outer housing of the controller 120 as shown in FIG. 1. Alternatively, the reservoir 110 may be provided separately.

Referring to FIG. 3, a side view of an ultrasound-based tissue treatment system is shown in accordance with an embodiment. In an embodiment, the catheter 102 can have a rapid-exchange type guidewire lumen 213. More particularly, the guidewire lumen 213 can extend from the distal tip of the catheter 102 through a partial length of the catheter shaft 214 to an exit port 225 in the distal portion 210 of the catheter 102. For example, a distance from the distal tip to the rapid exchange port may be in a range of 20 to 30 cm, e.g., 23 cm. The proximal hub 240 illustrated in FIG. 3 may differ from the proximal hub 240 illustrated in FIG. 2, given that the exit port may be moved from the proximal portion 220 to the distal portion 210. Other components of rapid exchange version of the catheter 102 may be similar to those of the over-the-wire version of the catheter 102, and thus, the descriptions of the components illustrated in FIG. 2 can apply to similarly numbered components illustrated in FIG. 3.

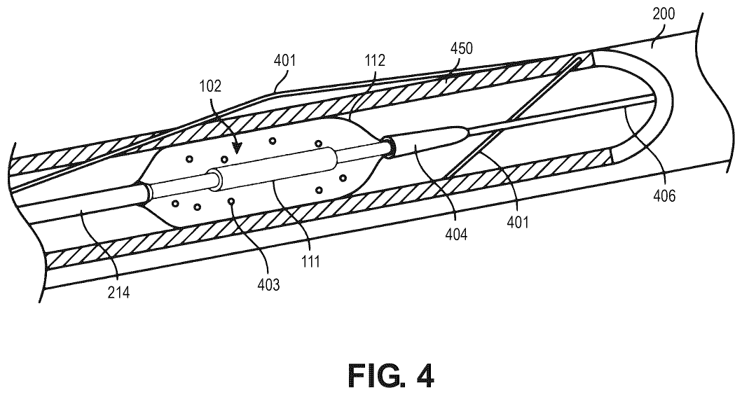

Referring to FIG. 4, a perspective view of an ultrasound-based tissue treatment system inserted into a body lumen is shown in accordance with an embodiment. Components of the distal portion 210 of the catheter 102 may be inserted into a body lumen of a subject. In FIG. 4, the body lumen is a blood vessel (e.g., a renal artery) that has a plurality of nerves 401 in an outer layer (e.g., adventitia layer) of the blood vessel. As described above, the distal portion 210 may include the ultrasound transducer 111, the balloon 112 filled with a cooling fluid 403, the catheter shaft 214, and/or a guidewire support tip 404 configured to receive a guidewire 406.

The transducer 111 may be disposed partially or completely within the balloon 112, which may be inflated with a cooling fluid 403 so as to contact the interior surface (e.g., intima) of the body lumen. In certain embodiments, the transducer 111 may be used to output an acoustic signal when the balloon 112 fully occludes a body lumen of a target vessel 200.

The balloon 112 may center the transducer 111 within the body lumen. In certain embodiments, e.g., suitable for renal denervation, the balloon 112 is inflated while inserted in the body lumen of the patient during a procedure at a working pressure of about 10 to about 30 psi using the cooling fluid 403. The balloon 112 may be or include a compliant, semi-compliant or non-compliant medical balloon. The balloon 112 is sized for insertion in the body lumen and, in the case of insertion into the renal artery, for example, the balloon 112 may be selected from available sizes including outer diameters of 3.5, 4.2, 5, 6, 7, or 8 mm, but not limited thereto.

In some embodiments, as shown in FIG. 4, when inflated by being filled with the cooling fluid 403 under the control of the controller 120, the outer wall of the balloon 112 may be generally parallel with the outer surface of the transducer 111. Optionally, the balloon 112 may be inflated sufficiently as to be in apposition with the body lumen. For example, when inflated, the balloon 112 may at least partially contact, and thus be in apposition with, an inner surface of a vessel wall 450 of the body lumen. When the balloon 112 is in apposition with the body lumen, and more specifically the interior circumferential wall of the body lumen, the balloon 112 can substantially stop blood within the body lumen from following past the balloon.

In other configurations, the balloon 112 is configured not to contact the body lumen when expanded. The balloon 112 may surround the transducer in order to cool the transducer during sonications, but the balloon may not contact or occlude the body lumen, and the blood within the body lumen may be relied upon to cool the body lumen instead of the cooling fluid. When the balloon 112 surrounds the transducer 111, but the balloon does not contact or occlude the body lumen, the balloon 112 may be non-compliant. In certain embodiments, the balloon 112 comprises nylon.

For a blood vessel that matches the balloon diameter, the non-compliant balloon (e.g., 112) can act as the centering mechanism. The cooling of the vessel wall can be managed by a cooling system of the generator by flowing water or other cooling fluid, such as dextrose or saline, through the balloon if necessary. A non-compliant balloon advantageously offers tighter control of balloon design. The non-compliant balloon (e.g., 112) may be advantageously constructed such that the balloon surface, which is without wrinkles when under inflation, does not interfere with sonication, and holds the desired shape. Additionally, or alternatively, the balloon 112 may be maintained at a specified size by pushing cooling fluid through and/or pulling cooling fluid out of the balloon 112 at a specified flow rate.

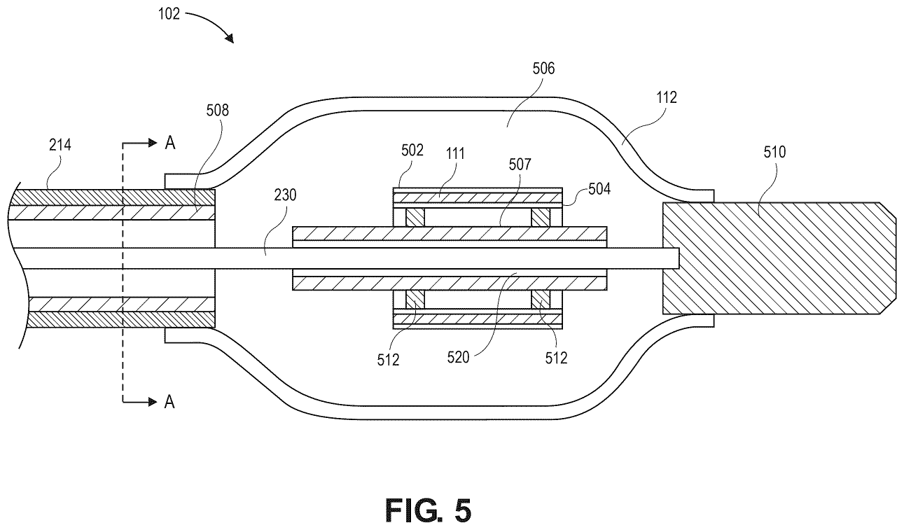

Referring to FIG. 5, a longitudinal cross-sectional view of a distal portion of a catheter of an ultrasound-based tissue treatment system is shown in accordance with an embodiment. The ultrasound transducer 111 may include a cylindrical hollow tube made of a piezoelectric material (e.g., lead zirconate titanate (PZT), etc.), with inner and outer electrodes 502, 504 disposed on the inner and outer surfaces of the cylindrical tube, respectively. Such a cylindrical hollow tube of piezoelectric material is an example of, and thus can be referred to as, a piezoelectric transducer body. The piezoelectric transducer body can have various other shapes and need not be hollow. In certain embodiments suitable, e.g., for renal denervation, the piezoelectric material, of which the piezoelectric transducer body is made, is lead zirconate titanate 8 (PZT8), which is also known as Navy III Piezo Material. Raw PZT transducers may be plated with layers of copper, nickel and/or gold to create electrodes on surfaces (e.g., the inner and outer surfaces) of the piezoelectric transducer body. Application of a voltage and alternating current across inner and outer electrodes 502, 504 causes the piezoelectric material to vibrate transverse to the longitudinal direction of the cylindrical tube and radially emit ultrasonic waves.

In an embodiment, the ultrasound transducer 111 can be positioned within an interior 506 of the balloon 112. The balloon 112 can have the interior 506 in fluid communication with a fluid lumen 508 of the catheter shaft 214. The fluid lumen 508 can convey cooling fluid 403 into the interior 506 to cool the transducer 111. More particularly, the balloon 112 can contain the transducer 111 within the interior 506 such that the transducer 111 is contacted and cooled by cooling fluid 403 that passes into the interior 506 from the fluid lumen 508.

As shown in FIG. 5, the ultrasound transducer 111 can be generally supported via a backing member or post 507. In certain embodiments, the backing member 507 comprises stainless steel coated with nickel and gold, wherein nickel is used as a bonding material between the stainless steel and gold plating. In certain embodiments suitable, e.g., for renal denervation, an outer diameter of the transducer 111 is about 1.5 mm, an inner diameter of the transducer 111 is about 1 mm, and the transducer 111 has a length of about 6 mm. Transducers having other inner diameters, outer diameters, and lengths, and more generally dimensions and shapes, are also within the scope of the embodiments described herein. Further, it is noted that the drawings in the figures are not necessarily drawn to scale, and often are not drawn to scale.

The backing member 507 may extend from the distal portion of the catheter shaft 214 to a distal tip 510 of the catheter 102. For example, the distal end of the backing member 507 may be positioned within an adjacent opening in the distal tip 510, and the proximal end of the backing member 507 may be moveably coupled to the distal portion of the catheter shaft 214 via the electrical cabling 230. In other embodiments, there is a gap between the distal end of the catheter shaft 214 and the proximal end of the ultrasound transducer 111.

In order to permit liquid cooling along both the inner and outer electrodes 502, 504, the backing member 507 may include one or more stand-off assemblies 512. The stand-off assemblies 512 may define one or more annular openings through which cooling fluid 403 may enter the space of the transducer 111 (which may be selectively insulated) between the backing member 507 and the inner electrode 504. Accordingly, the backing member 507 may serve as a fluid barrier between the cooling fluid 403 circulated within the balloon 112 and the lumen of the backing member 507 that receives the guidewire 406.

In accordance with certain embodiments, the stand-off assemblies 512 are electrically conductive, so as to electrically couple the inner electrode 504 of the ultrasound transducer 111 to the backing member 507. One or more conductors of the electrical cabling 230 may be electrically coupled to the backing member 507. Thus, as the controller 120 is activated, current may be delivered from the electrical cabling 230 to the inner electrode 504 of the ultrasound transducer 111 via the backing member 507 and the stand-off assemblies 512, which advantageously eliminates the need to couple the cabling 230 directly to the inner electrode 504 of the transducer 111. In other embodiments, the backing member 507 and the stand-off assemblies 512 are made of one or more electrical insulator material(s), or if made of an electrically conductive material(s) are coated with one or more electrical insulator material(s). In certain embodiments, one or more electrical conductors of the cabling 230 are directly coupled (e.g., soldered) to the inner electrode 504 of the transducer 111.

The backing member 507 may have an isolation tube disposed along its interior surface so as to prevent or reduce the likelihood of electrical conduction between the guidewire 406 and the backing member 507, for use in embodiments where such an electrical conduction is not desired. The isolation tube can be formed of a non-electrically conductive material (e.g., a polymer, such as polyimide), which can also be referred to as an electrical insulator. As illustrated in FIG. 5, the isolation tube may extend through the lumen of the backing member 507 within the transducer 111 toward the distal tip 510. In this manner, the transducer 111 is distally offset from the distal end of the catheter shaft 214.

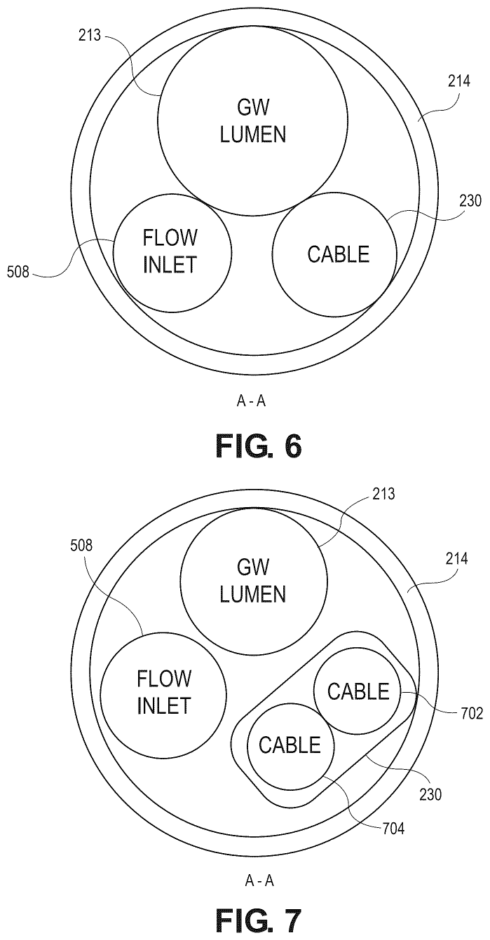

Referring to FIG. 6, a sectional view, taken about line A-A of FIG. 5, of a distal portion of catheter of an ultrasound-based tissue treatment system is shown in accordance with an embodiment. The catheter shaft 214 includes one or more lumens. For example, the catheter shaft 214 may include the fluid lumen 508 for transferring the inflation/cooling fluid, e.g., water, sterile water, saline, 5% dextrose (D5W), other liquids or gases, etc., from and/or to a fluid source, e.g., the reservoir 110, at the proximal region 220 of the catheter 102 external to the patient. In an embodiment, the catheter shaft 214 includes a single fluid channel to move fluid toward the balloon 112. For example, the fluid channel can deliver the inflation fluid from the fluidic port 234 to the balloon 112 under control of the controller 120. Accordingly, the inlet channel is in fluid communication with the balloon 112 to pass fluid through the balloon 112 at a flow rate selected to inflate the balloon 112. The flow rate also controls heat transfer between the balloon 112 and the vessel wall to reduce a likelihood of overheating tissue during treatment. For example, the flow rate can provide for active cooling of about the first millimeter of tissue to preserve the integrity of, e.g., the renal arterial wall.

In an embodiment, the catheter shaft 214 includes a guidewire lumen 213. The guidewire lumen 213 can extend through the catheter shaft 214 and, optionally, through the transducer 111. Furthermore, the guidewire lumen 213 can extend through the distal tip 510. Accordingly, the distal tip 510 can be tracked over a guidewire 406 to navigate through a patient anatomy. As described above, the catheter 102 can include electrical cabling 230. The electrical cabling 230 can be a single electrical cable extending longitudinally through the catheter shaft 214.

Referring to FIG. 7, a sectional view, taken about line A-A of FIG. 5, of a distal portion of catheter of an ultrasound-based tissue treatment system is shown in accordance with an embodiment. The catheter shaft 214 can include the fluid lumen 508 and guidewire lumen 213, as described above. In an embodiment, the electrical cabling 230 includes a plurality of electrical cables. For example, a first electrical cable 702 and a second electrical cable 704 can extend parallel to each other within a sheathing.

In an embodiment, the catheter 102 includes one-way fluid flow of cooling fluid 403 from the controller 120 through the balloon 112 to a surrounding environment. When the cooling fluid 403 passes through the balloon 112, the fluid cools the transducer 111 and transfers heat in the downstream direction of the blood vessel. The catheter 102 can include a flow control device to regulate the flow rate of cooling fluid 403 into the surrounding environment. It will be appreciated that a difference in flow rate between cooling fluid 403 entering the balloon 112 and cooling fluid 403 exiting the balloon 112 can influence balloon inflation. For example, when the entering flow rate is higher than the exiting flow rate, the balloon 112 will inflate, and vice versa. Accordingly, the flow control device can be used to control inflation of the balloon 112.

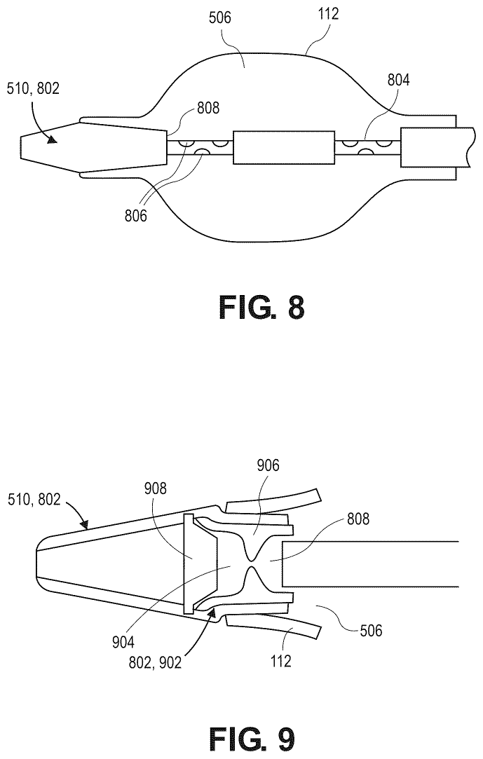

Referring to FIG. 8, a side view of a distal portion of a catheter of an ultrasound-based tissue treatment system having a flow control device is shown in accordance with an embodiment. In an embodiment, the catheter 102 includes a flow control device 802. The flow control device 802 can be located at the distal tip 510 of the catheter 102. For example, the flow control device 802 can be mounted on an inner member 804 of the catheter shaft 214. The inner member 804 may include one or more hole 806 in fluid communication with the fluid lumen 508. More particularly, the fluid lumen 508 can be coupled to a lumen of the inner member 804 to pass cooling fluid 403 through the hole(s) 806 of the inner member 804 into the interior 506 of the balloon 112.

The flow control device 802 may also be coupled to the balloon 112. More particularly, the balloon 112 can be sealed to an outer surface of the flow control device 802. Accordingly, cooling fluid 403 can flow from the interior 506 of the balloon 112 into the flow control device 802 and distally into a surrounding environment. Flowing cooling fluid 403 through the balloon interior 506 to the surrounding environment, rather than directly through the fluid lumen 508, can facilitate cooling fluid exchange within the balloon interior to enhance transducer and/or tissue cooling. For example, cooling fluid 403 that has been warmed by sonication in the balloon 112 can exit and be replenished with fresh cooling fluid. Such replenishment can occur without the need for a return lumen, allowing the overall device size to be reduced.

As described below, the flow control device 802 can provide one-way fluid flow for providing variable pressure to the balloon 112, e.g., a compliant balloon. The flow control device 802 can have an inlet port 808 to receive fluid passing through the interior 506 from the fluid lumen 508. The flow control device 802 can similarly have an outlet port, e.g., in a distal or side surface of the device, to pass fluid outward to the surrounding environment. Fluid flow through the flow control device 802 may occur when the fluid reaches a predetermined pressure. More particularly, flow through the flow control device 802 may be regulated to turn on when the fluid pressure is above a threshold, and to turn off when the fluid pressure is below the threshold. Accordingly, the flow control device 802 is configured to pass the fluid to the surrounding environment when the fluid has the predetermined pressure.

Referring to FIG. 9, a sectional view of a flow control device of a catheter of an ultrasound-based tissue treatment system is shown in accordance with an embodiment. The flow control device 802, which allows for a second fluid lumen to be omitted by venting cooling fluid 403 rather than circulating cooling fluid 403, can control the exit flow using a one-way valve. In an embodiment, the flow control device 802 includes a check valve 902. The check valve 902 can be located in the distal tip 510 of the catheter 102, and a pressure, e.g., a cracking pressure, on an input side of the valve that is higher than a pressure on the output side of the valve, e.g., an outlet port 904, can cause the check valve 902 to open. When the valve is open, the cooling fluid 403 can pass through the valve and the distal tip 510 of the catheter 102 into the surrounding environment. By contrast, when the input side pressure is below the cracking pressure, the valve can close and fluid will not pass through the valve and the distal tip 510 into the surrounding environment.

The check valve 902 can include the inlet port 808 to receive fluid from the interior 506 of the balloon 112. The inlet port 808 can carry fluid toward an elastomeric valve 906 of the check valve 902. The elastomeric valve 906 may, for example, be a duckbill valve, a slit membrane, etc. The elastomeric valve 906 can be backed by a sealing plunger 908. By backing the elastomeric valve 906, the sealing plunger 908 can maintain a shape of the elastomeric valve 906, e.g., prevent deformation of the elastomeric valve 906 that would cause unwanted venting of the fluid.

In an embodiment, the elastomeric valve 906 can be formed from soft polyurethane, e.g., Shore A polyurethane, and can recover consistently when opened and closed under pressure. The elastomeric valve 906 can have a crack pressure equal to the predetermined pressure at which the check valve 902 is meant to open. For example, the predetermined pressure may be a desired inflation pressure of the balloon 112. The desired inflation pressure may be a pressure required to inflate the balloon 112 to a predetermined, corresponding diameter. Accordingly, when the fluid pressure within the balloon interior 506 is at the predetermined pressure, the balloon 112 can have the predetermined diameter, and the elastomeric valve 906 can open to pass cooling fluid 403 into the surrounding environment to both cool the transducer 111 and maintain the balloon at the predetermined diameter.

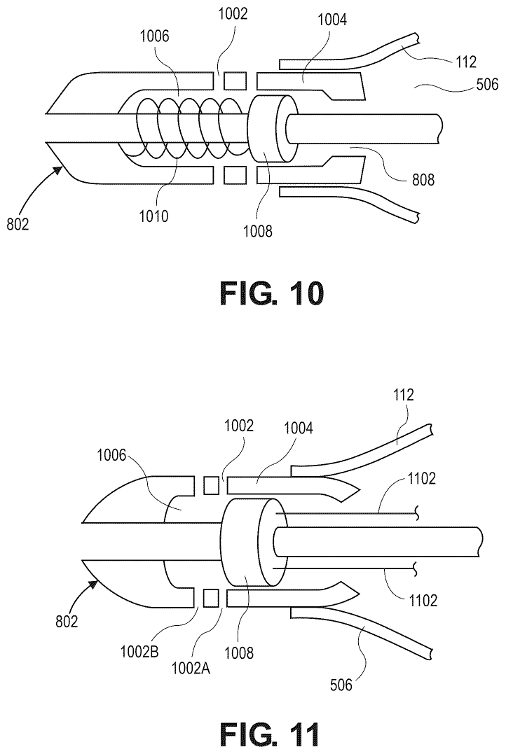

Referring to FIG. 10, a sectional view of a flow control device of a catheter of an ultrasound-based tissue treatment system is shown in accordance with an embodiment. The flow control device 802 can include one or more vent holes 1002 to vent or dump the cooling fluid 403 into the surrounding environment. The vent hole(s) 1002 can extend through an outer wall 1004 of the flow control device 802. More particularly, the flow control device 802 can have an outer wall 1004 surrounding an internal channel 1006, and the vent hole 1002 can extend from the internal channel 1006, through the outer wall 1004, into the surrounding environment. Accordingly, the vent hole 1002 provides an exit path for the cooling fluid 403.

The internal channel 1006 may be in fluid communication with the interior 506 of the balloon 112 via the inlet port 808. Cooling fluid 403 may therefore flow from the interior 506 through the inlet port 808 toward the vent hole(s) 1002. In an embodiment, however, fluid flow is regulated by a plunger 1008. The plunger 1008 can be movable within the internal channel 1006 relative to the vent hole 1002. For example, the plunger 1008 can include an annular body riding over the guidewire lumen 213. Movement of the plunger 1008 may be influenced by other components of the flow control device 802. For example, the flow control device 802 may include a spring 1010 to bias the plunger 1008 relative to the vent hole 1002.

The spring 1010 can bias the plunger 1008 in a direction, e.g., proximally, within the internal channel 1006. In the biased position, the plunger 1008 can be proximal to the vent hole 1002, and may seal against the outer wall 1004 to prevent fluid from flowing through the vent hole 1002. As the fluid pressure increases, however, the fluid pressure can press against the plunger 1008 against the biasing force of the spring 1010. When the fluid pressure reaches the predetermined pressure, the biasing force of the spring 1010 can be overcome and the plunger 1008 can slide distally, compressing the spring 1010. In this actuated position, the plunger 1008 can be distal to the vent holes 1002. Accordingly, cooling fluid 403 may flow into the internal channel 1006 and through the vent hole(s) 1002 proximal to the plunger 1008, into the surrounding environment. The fluid flow rate and/or pressure can be automatically adjusted by the spring 1010.

Referring to FIG. 11, a sectional view of a flow control device of a catheter of an ultrasound-based tissue treatment system is shown in accordance with an embodiment. A position of the plunger 1008 relative to the vent hole 1002 may be adjusted using one or more pull wires 1102. A pull wire 1102 can connect to the plunger 1008, e.g., by a thermal or adhesive bond, and may extend proximally through the catheter 102 to a proximal end. The proximal end may be coupled to an actuation member, such as a lever, knob, etc., to allow a user to pull and/or push on the pull wire 1102. Pulling or pushing the pull wire 1102 can control a location of the plunger 1008 relative to the vent hole 1002. More particularly, the pull wire 1102 can move the plunger 1008 relative to the vent hole 1002. The fluid flow rate and/or pressure can be manually adjusted by the pull wire 1102.

The flow control device 802 can have several vent holes 1002, and a position of the plunger 1008 can determine an amount of fluid flow through the vent holes 1002. For example, the several vent holes 1002 may be staggered in an axial direction, e.g., a first vent hole 1002A offset distally from a second vent hole 1002B. The plunger 1008 may slide past the first vent hole 1002A and expose the first vent hole 1002A to the cooling fluid 403, while still occluding the second vent hole 1002B. Accordingly, cooling fluid 403 may flow through the first vent hole 1002A and not the second vent hole 1002B when the plunger 1008 is at a first position. The plunger 1008 may subsequently slide past the second vent hole 1002B and expose both vent holes 1002 to the cooling fluid 403. Accordingly, cooling fluid 403 may flow through both vent holes 1002A and 1002B. The plunger position can therefore control an open venting area for dumping cooling fluid 403 into the surrounding environment, and thus, an amount of fluid passing through the vent holes 1002. Altering the exit flow of the cooling fluid 403 may in turn influence a pressure of the cooling fluid 403 within the interior 506 of the balloon 112.

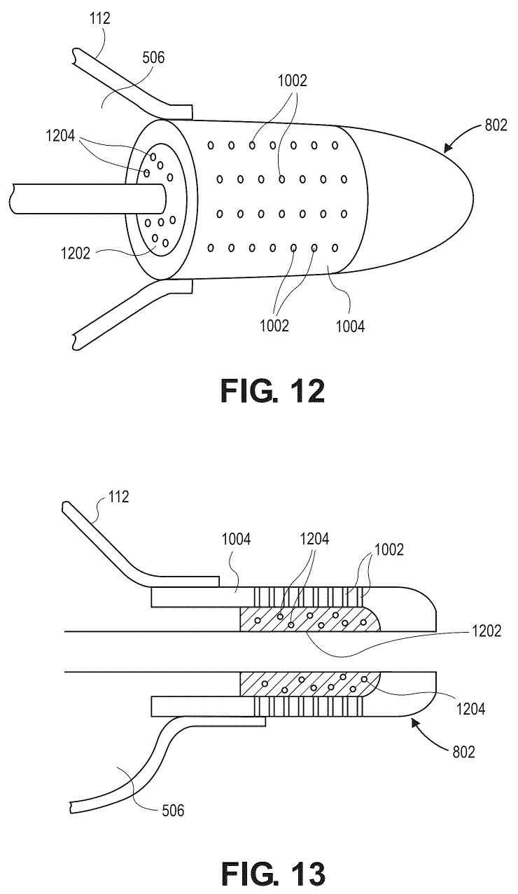

Referring to FIG. 12, a side view of a flow control device of a catheter of an ultrasound-based tissue treatment system is shown in accordance with an embodiment. The flow control device 802 may include a plurality of orifices that crack (open) at the predetermined fluid pressure. For example, in an embodiment, the flow control device 802 includes several vent holes 1002 to leak the cooling fluid 403 having the predetermined pressure. The vent holes 1002 can extend through the outer wall 1004 of the flow control device 802. Each hole can be in fluid communication with the interior 506 of the balloon 112, and can have a crack pressure to allow the flow of cooling fluid 403 when the fluid pressure within the interior 506 reaches the predetermined pressure.

The crack pressure of each hole may be the same. Each hole can have a same shape and size such that the resistance to flow of each hole is similar. Accordingly, the vent holes 1002 can weep fluid at a constant flow state. More particularly, the vent holes 1002 can each weep cooling fluid 403 into the surrounding environment at a same rate.

The crack pressure of each hole, or each group of several groups of hole, may be different. For example, some of the vent holes 1002 may have a smaller diameter than other vent holes 1002. The smaller vent holes 1002 may have higher flow impedance, and thus, may have higher crack pressures than the larger vent holes 1002. Flow may selectively pass through the larger holes 1002 at a first inflation pressure, and then pass through both the larger and smaller holes 1002 when the fluid pressure exceeds the crack pressure of the smaller holes 1002. The vent holes 1002 can therefore weep more fluid as the inflation pressure increases to maintain the balloon inflation pressure within a desired range above the predetermined pressure.

In an embodiment, the flow control device 802 includes a plug 1202. The plug 1202 can have several holes 1204 passing axially and/or radially through the plug body. For example, the plug 1202 may be formed from a semi-permeable material, such as an electro-spun fibrous material. The holes and/or channels formed in the plug 1202 can receive cooling fluid 403 from the interior 506 of the balloon 112 and pass the cooling fluid 403 distally and outward toward the vent holes 1002 of the flow control device 802. Accordingly, the plug 1202 can provide some resistance to the distal flow of the cooling fluid 403, however, can transmit the cooling fluid 403 toward the vent holes 1002 for dumping into the surrounding environment.

Referring to FIG. 13, a sectional view of a flow control device is shown in accordance with an embodiment. In cross-section, a network of channels formed by the holes 1204 is evident. The network of channels can convey cooling fluid 403 from the interior 506 of the balloon 112 to the vent holes 1002 in the outer wall 1004 of the flow control device 802. Accordingly, fluid can be vented from the interior 506 when the balloon 112 is inflated to a pressure that exceeds the crack pressure, e.g., the combined impedance, of the network of channels and the vent holes 1002.

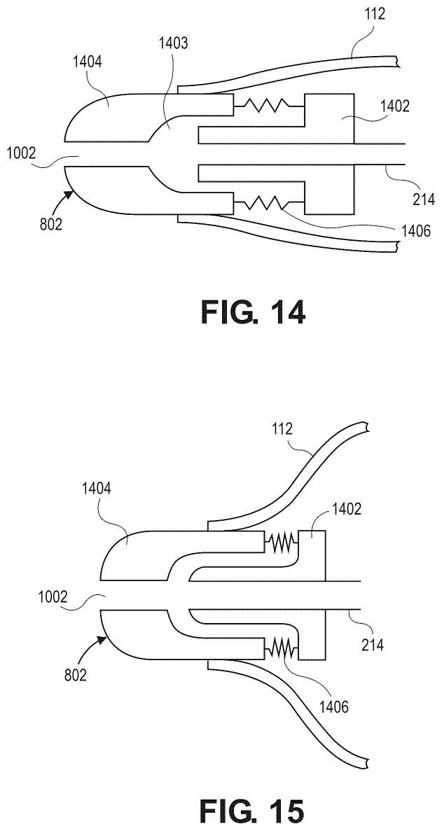

Referring to FIG. 14, a sectional view of a flow control device of a catheter of an ultrasound-based tissue treatment system is shown in accordance with an embodiment. The flow control device 802 may allow for relative movement between a distal end of the balloon 112 and the catheter shaft 214. Such movement can groom the balloon 112. More particularly, by allowing the distal end of the balloon 112 to move relative to the catheter shaft 214, the balloon 112 can be stretched and therefore reduced in profile. When the balloon 112 is stretched and reduced in profile, it may more easily track through the target anatomy. The reduced profile can allow the balloon 112 to track through narrow vessels, such as the radial artery, during delivery and/or retrieval.

In an embodiment, a self-grooming balloon 112 includes a flow control device 802 having a proximal tip portion 1402 and a distal tip portion 1404. The balloon 112 can be self-grooming because it may be biased to wrap or adjust, e.g., stretch, into a lower profile when fluid pressure within the balloon 112 is removed. For example, the balloon 112 may be manufactured in a wrapped and/or stretched configuration, and the balloon 112 may preferentially return to that configuration when deflated, based on a biasing force provided by the flow control device 802. When the balloon 112 is in the lower profile, it can be tracked through a narrow channel or vessel. For example, the balloon 112 can be delivered through a radial artery, or can be retracted into a guide sheath, without hanging up on or damaging the adjacent structure.

The proximal tip portion 1402 can be independent and movable relative to the distal tip portion 1404. For example, the proximal tip portion 1402 can be mounted on the catheter shaft 214, and the distal tip portion 1404 can be movably mounted on the proximal tip portion 1402. Whereas the proximal tip portion 1402 may be stationary relative to the catheter shaft 214, the distal tip portion 1404 may be coupled to the balloon 112. Accordingly, when the distal tip portion 1404 moves relative to the proximal tip portion 1402, the distal end of the balloon 112 can move relative to the catheter shaft 214.

In an embodiment, the distal tip portion 1404 is slidable on the proximal tip portion 1402. The distal tip portion 1404 can have a recess 1403 extending into a proximal end of the portion. The recess 1403 can be sized to receive a distal protrusion of the proximal tip portion 1402. More particularly, the protrusion of the proximal tip portion 1402 can extend into the recess 1403 of the distal tip portion 1404. Accordingly, the distal tip portion 1404 can move axially and be constrained radially by the proximal tip portion 1402. The distal tip portion 1404 may relate to the proximal tip portion 1402 essentially as a collar is movable and constrained by a shaft.

The flow control device 802 can include a spring 1406 to apply a counterforce to the distal tip portion 1404, thereby biasing the distal tip portion 1404 relative to the proximal tip portion 1402. The counterforce can be counter to a proximal load applied to the distal tip portion 1404 by the balloon 112. As the balloon 112 inflates, the balloon wall expands radially outward causing a length of the balloon to decrease. As the balloon 112 grows, the distal end of the balloon 112 will pull proximally on the distal tip portion 1404. The proximal load can act in a different direction than the biasing force of the spring 1406. More particularly, the spring 1406 can be a compression spring biasing the distal tip portion 1404 away from the proximal tip portion 1402. When the fluid pressure within the interior 506 of the balloon 112 is at a lower level, the spring force of the spring can overcome the retracting force applied to the distal tip portion 1404 by the distal end of the balloon 112. The spring 1406 can therefore expand and maintain the balloon in a small profile.

Referring to FIG. 15, a sectional view of a flow control device of a catheter of an ultrasound-based tissue treatment system is shown in accordance with an embodiment. When the fluid pressure within the interior 506 of the balloon 112 is at a higher level, the spring force of the spring 1406 may be insufficient to overcome the retracting force applied to the distal tip portion 1404 by the distal end of the balloon 112. As the balloon 112 grows, the distal tip portion 1404 can be pulled proximally, translating relative to the proximal tip portion 1402. The telescoping motion can compress the spring 1406 between the distal tip portion 1404 and the proximal tip portion 1402. For example, the distal tip portion 1404 can slide over the proximal tip portion 1402, reducing a gap between a proximal end of the distal portion and an adjacent wall of a base of the proximal tip portion 1402.

When the fluid pressure within the balloon 112 decreases from the higher level, e.g., due to venting of the cooling fluid 403 through the vent hole 1002 of the distal tip 510, the spring 1406 can again overcome the proximal force applied by the balloon 112 and return the distal tip portion 1404 distally away from the proximal tip portion 1402. The flow control device 802 may therefore be a self-grooming device, which both vents cooling fluid 403 to the surrounding environment, and stretches the balloon 112 after balloon deflation to move the balloon to a profile suitable to tracking through narrow vessels.

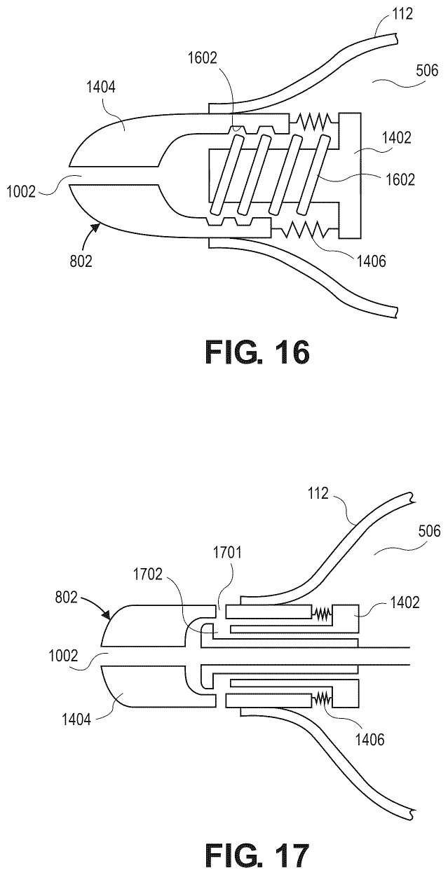

Referring to FIG. 16, a sectional view of a flow control device of a catheter of an ultrasound-based tissue treatment system is shown in accordance with an embodiment. In an embodiment, the distal tip portion 1404 of the flow control device 802 may be rotatable on the proximal tip portion 1402. For example, the portions, may have mating threads, e.g., a helical external thread on the proximal tip portion 1402 engaged with an internal thread of the distal tip portion 1404, to allow the portions to be screwed relative to each other. More particularly, the distal tip portion 1404 can be threadably coupled to the proximal tip portion 1402. When the proximal load of the balloon 112 on the distal tip portion 1404 pulls the portion proximally, the threads can cause the distal tip portion 1404 to slidably rotate over the proximal tip portion 1402. The balloon 112 may be twisted in a low-profile state prior to balloon inflation, and thus, the rotation of the distal tip portion 1404 can unwind the balloon 112 to an un-twisted state when the balloon inflates. Similarly, when the cooling fluid 403 within the balloon 112 is vented to the surrounding environment and the balloon deflates, the distal tip portion 1404 can be biased forward by the spring 1406 and rotated in an opposite direction causing the balloon 112 to twist. The twisted balloon can be wrapped into a low profile suitable for tracking through narrow vessels.

Referring to FIG. 17, a sectional view of a flow control device of a catheter of an ultrasound-based tissue treatment system is shown in accordance with an embodiment. The flow control device 802 may include a self-grooming device that incorporates one or more actuatable vent holes 1002. The distal portion may have a vent hole 1002 extending along a central axis of the flow control device 802 to vent fluid from the balloon 112, similar to the embodiments described with respect to FIGS. 14-16. The axial vent hole 1002 may be always open, and may vent fluid whenever cooling fluid 403 is circulated to the balloon 112. Additionally, the distal portion can include a side vent hole 1701, extending radially outward relative to the central axis. The distal tip portion 1404, and thus the side vent hole 1701, can move relative to the proximal tip portion 1402.

In an embodiment, the proximal tip portion 1402 includes a fluid port 1702. The fluid port 1702 can extend through the proximal tip portion 1402 from an inlet in fluid communication with the interior 506 of the balloon 112 to an outlet at a lateral surface of the proximal tip portion 1402. For example, the fluid port 1702 can face laterally outward from the protrusion of the proximal tip portion 1402 that slides within the distal tip portion 1404. Accordingly, the fluid port 1702 outlet can face toward the side vent hole 1701 when the fluid port 1702 and the vent hole 1002 are axially aligned. More particularly, the distal tip portion 1404 can be movable relative to the proximal tip portion 1402 to align the side vent hole 1701 with the fluid port 1702. In the actuated state, shown in FIG. 17, when the fluid port 1702 and the side vent hole 1701 are aligned, cooling fluid 403 can flow through the fluid port 1702 and the side vent hole 1701 to the surrounding environment. Notably, fluid flow can be established in the actuated state when the balloon 112 is inflated, pulling the distal tip portion 1404 proximally and compressing the spring 1406.

When fluid exits the vent hole 1002 through the lateral wall of the distal tip portion 1404, pressure can be relieved in the balloon 112, and the spring 1406 may bias the distal tip portion 1404 away from the proximal tip portion 1402. The flow control device 802 can be biased to an actuated state, in which the fluid port 1702 and the side vent hole 1701 are misaligned. The distal tip portion 1404 and the proximal tip portion 1402 can be engaged in a sliding fit that has insufficient gap between the components to allow for fluid flow when the fluid port 1702 and the vent hole 1002 are misaligned. Accordingly, cooling fluid 403 can only vent through the lateral vent hole 1701 in the actuated state. The lateral vent hole 1701 can therefore be actuated when balloon pressure rises above a threshold limit to quickly vent cooling fluid 403 and lower the balloon pressure to the predetermined pressure. At the predetermined pressure, the balloon 112 can have a predetermined inflation diameter.

Actuation of the flow control device to move the distal tip portion 1404 relative to the proximal tip portion 1402 is automatically controlled in the embodiments described above. More particularly, the pressure within the balloon 112 applies a retroactive load to the distal tip portion 1404 that acts against the spring bias. The spring 1406 and the balloon 112 therefore operate in combination to determine whether the flow control device 802 is actuated. In an embodiment, however, actuation of a flow control device 802 is performed manually. For example, the catheter 102 may include a pull wire that extends from a handle at a proximal end of the device to the distal tip portion 1404. The pull wire can retract the distal tip portion 1404 relative to the proximal tip portion 1402, or be pushed to advance the distal tip portion 1404 over the proximal tip portion 1402. Relative movement of the distal tip portion 1404 and the proximal tip portion 1402 can control the flow of cooling fluid 403 and/or groom balloon 112, as described above.

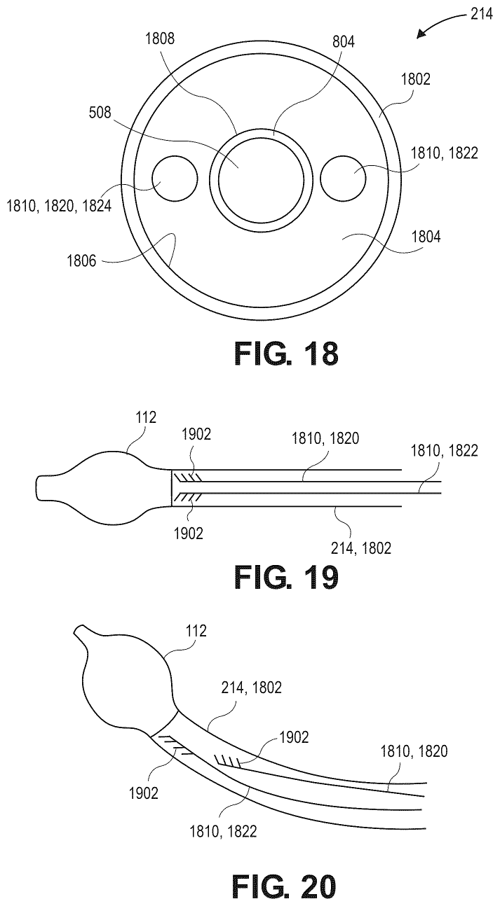

Referring to FIG. 18, a sectional view of a catheter shaft of a catheter of an ultrasound-based tissue treatment system is shown in accordance with an embodiment. As described above, the catheter shaft 214 can have a shaft diameter less than or equal to 5 French to facilitate radial access. To achieve such a diameter, the catheter shaft 214 may not include a guidewire lumen 213. More particularly, the omission of a guidewire lumen 213 can reduce the components of the catheter shaft 214, and thus, an overall dimension of the catheter shaft 214 can be reduced. Without a guidewire lumen 213 to track the catheter 102 over guidewire 406, however, an alternative manner of steering the device through the vasculature may be needed. Accordingly, the catheter 102 may be configured as a steerable catheter.

In an embodiment, the catheter shaft 214 includes an outer member 1802 having a central lumen 1804. The outer member 1802 can include a tubular structure having an inner wall 1806 containing the central lumen 1804. In an embodiment, the inner member 804 of the catheter 102 can extend through the central lumen 1804. For example, the inner member 804 can include a tubular structure extending from a proximal end of the catheter 102 to the distal end of the catheter 102, e.g., through the balloon 112. The inner member 804 can have a fluid lumen 508 to deliver fluid to the interior 506 of the balloon 112. For example, the inner member 804 can include hole 806 to diffuse cooling fluid 403 into the interior 506 of the balloon 112 (FIG. 8). A wall 1808 of the inner member 804 provides the tubular structure having an outer wall 1808 facing radially outward toward the inner wall 1806 of the outer member 1802. Accordingly, the outer member 1802 and the inner member 804 may be coaxial tubular structures forming a gap between the outer wall 1808 and the inner wall 1806.

The gap between the inner member 804 and the outer member 1802 may be a portion of the central lumen 1804 through which one or more steering wires 1810 extend. More particularly, the steering wire(s) 1810 can extend through the central lumen 1804 between the inner wall 1806 of the outer member 1802 and the outer wall 1808 of the inner member 804. The steering wires 1810 may be pulled or pushed, e.g., via actuation of handle elements, to steer the catheter 102.

In an embodiment, the catheter 102 includes a first steering wire 1820 extending through the central lumen 1804. The catheter 102 may also include a second steering wire 1822 extending through the central lumen 1804. The first steering wire 1820 may be diametrically opposite the second steering wire 1822 within the central lumen 1804. More particularly, the first steering wire 1820 may be on a first lateral side of the inner member 804, and the second steering wire 1822 may be on a second lateral side of the inner member 804, opposite the first steering wire 1820.

The steering wires 1810 may be formed from metallic or polymeric elongated elements, such a polyimide wire. In an embodiment, at least one of the one or more steering wires 1810 may be an electrical cable 1824. The electrical cable 1824 can extend to the distal end of the catheter 102 to deliver energy to the ultrasound transducer 111.

Referring to FIG. 19, a side view of a catheter of an ultrasound-based tissue treatment system is shown in accordance with an embodiment. The one or more steering wires 1810 can connect to the outer member 1802 or the inner member 804 of the catheter shaft 214. More particularly, each steering wire 1810 can attach to the catheter shaft 214 at a respective anchor point 1902. The anchor points 1902 may, for example, be located proximal to the balloon 112.

Referring to FIG. 20, a side view of a catheter of an ultrasound-based tissue treatment system is shown in accordance with an embodiment. Pulling on the steering wires 1810 can transmit force to the anchor points 1902, and thus, to the catheter shaft 214. When the first steering wire 1820 is pulled, for example, the outer member 1802 is compressed along the lateral side adjacent to the first steering wire 1820, and thus, the catheter deflects, e.g., bends, in a direction of the lateral side. Similarly, pulling the second steering wire 1822 can steer the catheter 102 in the opposite direction, toward the lateral side adjacent to the second steering wire 1822. The steering wires 1810 can be bi-directional, and may be pushed to steer the catheter 102. More particularly, applying loads to the catheter 102 through the steering wires 1810 by pushing or pulling the wires can steer the catheter 102. Accordingly, the catheter 102 can be tracked through the vasculature to navigate to the target anatomy without the aid of a guidewire.

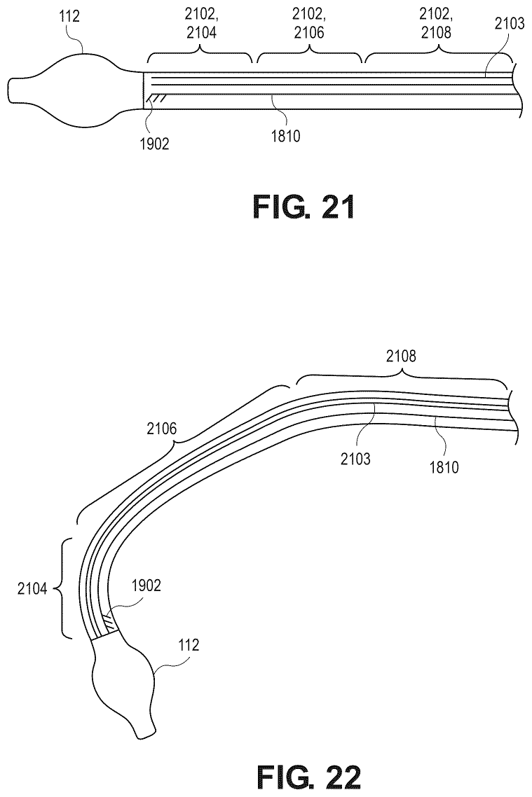

Referring to FIG. 21, a side view of a catheter of an ultrasound-based tissue treatment system is shown in accordance with an embodiment. A reduction in an overall dimension of the catheter system being introduced into the patient anatomy may also be facilitated by the omission of a guide sheath. The guide sheath may typically be used to introduce the catheter 102 to a particular anatomy, and can have a complex curvature that facilitates delivery to that anatomy. The guide sheath, however, includes a wall thickness that can add to the overall dimension of the catheter system. Removing the guide sheath can therefore reduce the overall dimension and allow the catheter system to more easily access certain anatomies.

In an embodiment, the catheter 102 is configured to deflect from a straightened configuration to a curved configuration. In the curved configuration, the catheter 102 can have a complex curvature. More particularly, various sections of the catheter 102 may curve differently such that an overall profile of the curved catheter shaft 214 exhibits varied curvatures from the proximal end of the catheter 102 to the distal end of the catheter 102. The catheter shaft 214 can include several shaft sections 2102. Each shaft section 2102 can have a respective stiffness. For example, the shaft sections 2102 can include a distal section 2104, an intermediate section 2106, and a proximal section 2108. By way of example, the distal section 2104 can include a most distal 1-3 cm, e.g., 2 cm, length of the catheter shaft 214 extending proximally from the balloon 112, the intermediate section 2106 can include a 3-5 cm, e.g., 4 cm, length of the catheter shaft 214 extending proximally from the distal section 2104, and the proximal section 2108 can include a balance of the catheter shaft length extending proximally from the intermediate section 2106. The stiffnesses of each of those sections may differ and increase in the distal direction. Accordingly, the respective stiffness of the distal section 2104 may be less than the respective stiffness of the intermediate section 2106, and the respective stiffness of the intermediate section 2106 may be less than the respective stiffness of the proximal section 2108.

In an embodiment, the catheter 102 includes a steering wire 1810 attached to the catheter shaft 214 at an anchor point 1902. The anchor point 1902 may be at the distal end of the catheter shaft 214, e.g., within the distal section 2104 of the catheter 102. The steering wire 1810 may have a structure similar to that described above with respect to FIGS. 18-20.

The stiffness of the catheter 102 may also be influenced by a center support 2103. The center support 2103 can be a stiffening member that extends through the central lumen 1804 of the catheter shaft 214. The center support 2103 may, for example, be a hypotube. The hypotube can have a semi-rigid construction, and may have sufficient stiffness to contribute pushability to the catheter shaft 214.

Referring to FIG. 22, a side view of a catheter of an ultrasound-based tissue treatment system is shown in accordance with an embodiment. The variable stiffness of the catheter shaft 214 can cause the catheter 102 to take a complex curvature when the steering wire 1810 is pulled. Pulling the steering wire 1810 can exert a deflecting load at the anchor point 1902 that causes the catheter 102 to bend throughout the shaft sections 2102. The shaft can have discrete angular changes at the transitions between the sections. More particularly, a radius of curvature of the proximal section 2108 may be greater than a radius of curvature at the intermediate section 2106, and the radius of curvature of the intermediate section 2106 may be greater than a radius of curvature of the distal section 2104 when the catheter 102 is deflected. The changes in deflection curvature from section to section may be based on the stiffness of the outer member 1802, or the inner member 804, over the sections.

Changes in catheter stiffness that contribute to the complex curvature may also be influenced by the center support 2103. In an embodiment, the center support 2103 includes the inner member 804. For example, the inner member 804 may be a hypotube to carry cooling fluid 403 to the balloon 112. The inner member 804 can have the wall 1808 (FIG. 18), and in an embodiment, the wall 1808 is thinner at the distal section 2104 than at the intermediate section 2106. For example, the hypotube may be centerless ground to change the wall thickness over its length. The thinner wall 1808 over the distal section 2104 can make the distal section 2104 more flexible than the intermediate section 2106 or the proximal section 2108. Accordingly, like the changes in stiffness in the outer member 1802 described above, the changes in stiffness of the inner member 804 may cause sections to deflect differently and produce a complex curvature when the catheter is steered. The complex curvature may have the shape that is otherwise provided by a guiding sheath, and thus, the catheter 102 may be used in a radial access approach without the need for such a guiding sheath.

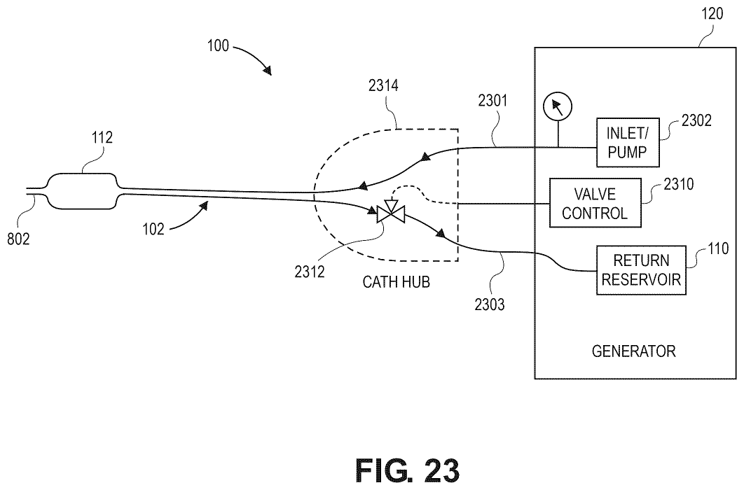

Referring to FIG. 23, a schematic view of an ultrasound-based tissue treatment system is shown in accordance with an embodiment. In an embodiment, the system incorporates a hybrid flow configuration, in which cooling fluid 403 is vented to the surrounding environment through the distal end of the catheter 102, and cooling fluid 403 is also returned to the controller 120. The catheter 102 may therefore include an inlet line 2301, to convey cooling fluid 403 from an inlet pump 2302 of the controller 120 to the balloon 112, and an outlet line 2303, to convey cooling fluid 403 from the balloon 112 to the controller 120, e.g., to the reservoir 110. The catheter 102 may also include the flow control device 802 to vent fluid to the surrounding environment. Accordingly, the hybrid flow system can convey more fluid to the balloon 112 than is required to be returned to the reservoir 110 via the outlet line 2303.

A flow of the fluid being circulated from/to the controller 120 may be regulated by a valve control 2310. For example, the valve control 2310 may be integrated in the controller 120, and can be electrically coupled to a flow valve 2312. The flow valve 2312 may be integrated in a catheter hub 2314 of the catheter 102. Alternatively, the flow valve 2312 may be integrated within the controller 120. The flow valve 2312 can be actuated by the valve control 2310 to open or close, thereby allowing or restricting flow from the balloon 112 to the reservoir 110.

When the flow valve 2312 is integrated in the catheter hub 2314, the flow valve 2312 may be actuated manually. For example, the catheter hub 2314 may have a switch, button, or knob to allow a user to manually adjust the valve state (open or close the valve). When the flow valve 2312 is closed, the balloon 112 can inflate. When the flow valve 2312 is open, the balloon 112 may deflate. Accordingly, the user can manually control balloon inflation diameter by actuating the flow valve 2312.

When some fluid is being dumped into the surrounding environment, and some cooling fluid 403 is being recirculated to the controller 120, then the rate of injection from the inlet pump 2302 and the return to the reservoir 110 can be regulated to control balloon inflation. For example, when the flow rate to the balloon 112 is equal to the venting flow rate plus the return flow rate, the balloon inflation diameter can remain constant. Increasing the inlet flow rate above the sum of the vented and returned flow rate can cause the balloon diameter to increase. By contrast, decreasing the inlet flow rate (or increasing the vented and returned flow rate) to cause more fluid to leave the balloon than enter the balloon can cause the balloon diameter to decrease. Accordingly, the inlet pump 2302, flow control device 802, and/or flow valve 2312 can be controlled to regulate balloon inflation and deflation.

In an embodiment, the outlet line 2303 can include a lumen to convey the cooling fluid 403 to the controller 120. The returned fluid rate can be less than the fluid flow rate to the balloon 112, as described above, and thus the outlet line 2303 may have a lumen that is smaller than the lumen carrying fluid to the balloon 112. More particularly, since less fluid is returned, the return lumen can be smaller. A reduction in the lumen size can translate to a reduced overall dimension of the catheter 102. Accordingly, the miniaturization of the return line can contribute to the catheter shaft 214 being sized for radial access.

On the other hand, the fluid lumens 508 of the catheter 102 may be sized to avoid blocking of the catheter shaft 214 during priming. Smaller lumens may become blocked by air bubbles that are generated during priming, and thus, it may be advantageous to increase the size of certain lumens that are susceptible to such blockage. In an embodiment, the return line has a lumen that is larger than the inlet line lumen. Accordingly, the return line can be resistant to blockage, e.g., may have a reduced likelihood of becoming blocked by air bubbles during priming.

In an embodiment, a catheter includes a catheter shaft, a balloon, an ultrasound transducer, and a flow control device. The catheter shaft has a fluid lumen. The balloon is mounted on the catheter shaft and has an interior in fluid communication with the fluid lumen. The ultrasound transducer is in the interior. The flow control device has an inlet port to receive fluid passing through the interior from the fluid lumen. The flow control device is configured to vent the fluid to a surrounding environment when the fluid has a predetermined pressure.

In an embodiment, the flow control device includes a check valve.

In an embodiment, the check valve includes a sealing plunger backing an elastomeric valve. The elastomeric valve has a crack pressure equal to the predetermined pressure.

In an embodiment, the flow control device includes a vent hole and a plunger.

The vent hole extends through an outer wall from an internal channel. The plunger is movable within the internal channel relative to the vent hole.

In an embodiment, the catheter includes a spring to bias the plunger relative to the vent hole.

In an embodiment, the catheter includes a pull wire to move the plunger relative to the vent hole.

In an embodiment, the flow control device includes a plug having several holes to leak the fluid having the predetermined pressure.

In an embodiment, the flow control device includes a proximal tip portion, a distal tip portion movable relative to the proximal tip portion, and a spring between the proximal tip portion and the distal tip portion.

In an embodiment, the proximal tip portion is mounted on the catheter shaft. The balloon is coupled to the distal tip portion.

In an embodiment, the distal tip portion is slidable on the proximal tip portion.

In an embodiment, the distal tip portion is rotatable on the proximal tip portion.

In an embodiment, the distal tip portion is threadably coupled to the proximal tip portion.

In an embodiment, the distal tip portion includes a vent hole. The proximal tip portion includes a fluid port. The distal tip portion is movable relative to the proximal tip portion to align the vent hole with the fluid port.

In an embodiment, the catheter shaft has a shaft diameter less than or equal to 5 French.

In an embodiment, a catheter includes a catheter shaft, a balloon, an ultrasound transducer, and a check valve. The catheter shaft has a fluid lumen. The balloon is mounted on the catheter shaft and has an interior in fluid communication with the fluid lumen. The ultrasound transducer in the interior. The check valve has an inlet port to receive fluid passing through the interior from the fluid lumen. The check valve is configured to vent the fluid to a surrounding environment when the fluid has a predetermined pressure.

In an embodiment, the check valve includes a sealing plunger backing an elastomeric valve. The elastomeric valve has a crack pressure equal to the predetermined pressure.

In an embodiment, a catheter includes a catheter shaft, a balloon, an ultrasound transducer, and a flow control device. The catheter shaft has a fluid lumen. The balloon is mounted on the catheter shaft and has an interior in fluid communication with the fluid lumen. The ultrasound transducer is in the interior. The flow control device has an inlet port to receive fluid passing through the interior from the fluid lumen. The flow control device includes a vent hole and a plunger. The vent hole extends through an outer wall from an internal channel. The plunger is movable within the internal channel relative to the vent hole. The flow control device is configured to vent the fluid to a surrounding environment.

In an embodiment, the catheter includes a spring to bias the plunger relative to the vent hole.

In an embodiment, the catheter includes a pull wire to move the plunger relative to the vent hole.

In an embodiment, a catheter includes a catheter shaft, a balloon, an ultrasound transducer, and a flow control device. The catheter shaft has a fluid lumen. The balloon is mounted on the catheter shaft and has an interior in fluid communication with the fluid lumen. The ultrasound transducer is in the interior. The flow control device has an inlet port to receive fluid passing through the interior from the fluid lumen. The flow control device includes a plug having several holes to leak the fluid to a surrounding environment when the fluid has a predetermined pressure.

In an embodiment, a catheter includes a catheter shaft, a balloon, an ultrasound transducer, and a flow control device. The catheter shaft has a fluid lumen. The balloon is mounted on the catheter shaft and has an interior in fluid communication with the fluid lumen. The ultrasound transducer is in the interior. The flow control device has an inlet port to receive fluid passing through the interior from the fluid lumen. The flow control device includes a proximal tip portion mounted on the catheter shaft, a distal tip portion coupled to the balloon and movable relative to the proximal tip portion, and a spring between the proximal tip portion and the distal tip portion. The flow control device is configured to vent the fluid to a surrounding environment when the fluid has a predetermined pressure.

In an embodiment, a catheter includes a catheter shaft, a balloon, an ultrasound transducer, and a flow control device. The catheter shaft has a fluid lumen. The balloon is mounted on the catheter shaft and has an interior in fluid communication with the fluid lumen. The ultrasound transducer is in the interior. The flow control device has an inlet port to receive fluid passing through the interior from the fluid lumen. The flow control device includes a proximal tip portion, a distal tip portion movable relative to the proximal tip portion, and a spring between the proximal tip portion and the distal tip portion. The flow control device is configured to vent the fluid to a surrounding environment when the fluid has a predetermined pressure.

In an embodiment, the distal tip portion is slidable on the proximal tip portion.

In an embodiment, the distal tip portion is rotatable on the proximal tip portion.

In an embodiment, the distal tip portion is threadably coupled to the proximal tip portion.

In an embodiment, the distal tip portion includes a vent hole. The proximal tip portion includes a fluid port. The distal tip portion is movable relative to the proximal tip portion to align the vent hole with the fluid port.

In an embodiment, a catheter includes a catheter shaft, a balloon, an ultrasound transducer, and a flow control device. The catheter shaft has a fluid lumen. The catheter shaft has a shaft diameter less than or equal to 5 French. The balloon is mounted on the catheter shaft and has an interior in fluid communication with the fluid lumen. The ultrasound transducer is in the interior. The flow control device has an inlet port to receive fluid passing through the interior from the fluid lumen. The flow control device is configured to vent the fluid to a surrounding environment when the fluid has a predetermined pressure.

In an embodiment, a catheter includes a catheter shaft, a balloon, an ultrasound transducer, and a flow control device. The balloon has an interior. The ultrasound transducer is in the interior. The catheter shaft includes an outer member having a central lumen, an inner member extending through the central lumen and having a fluid lumen to deliver fluid to the interior, and one or more steering wires extending through the central lumen between an inner wall of the outer member and an outer wall of the inner member. The one or more steering wires connect to the outer member or the inner member at an anchor point proximal to the balloon.

In an embodiment, the one or more steering wires includes a first steering wire diametrically opposite a second steering wire.

In an embodiment, one or more of the first steering wire or the second steering wire includes an electrical cable to deliver energy to the ultrasound transducer.

In an embodiment, the catheter shaft has several shaft sections. Each shaft section has a respective stiffness.

In an embodiment, the several shaft sections includes a distal section, an intermediate section, and a proximal section. The respective stiffness of the distal section is less than the respective stiffness of the intermediate section. The respective stiffness of the intermediate section is less than the respective stiffness of the proximal section.

In an embodiment, the inner member has a wall. The wall is thinner at the distal section than at the intermediate section.

In an embodiment, an ultrasound-based tissue treatment system includes any of the catheter embodiments described above, a controller, and a connection cable interconnecting the catheter and the controller.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

1-36. (canceled)

37. A catheter, comprising:
a catheter shaft having a fluid lumen;
a balloon mounted on the catheter shaft and having an interior in fluid communication with the fluid lumen;
an ultrasound transducer in the interior; and
a flow control device having an inlet port to receive fluid passing through the interior from the fluid lumen, wherein the flow control device is configured to vent the fluid to a surrounding environment when the fluid has a predetermined pressure.

38. The catheter of claim 37, wherein the flow control device includes a check valve.

39. The catheter of claim 38, wherein the check valve includes a sealing plunger backing an elastomeric valve, and wherein the elastomeric valve has a crack pressure equal to the predetermined pressure.

40. The catheter of claim 37, wherein the flow control device includes a vent hole extending through an outer wall from an internal channel, and a plunger movable within the internal channel relative to the vent hole.

41. The catheter of claim 40, further comprising a spring to bias the plunger relative to the vent hole.

42. The catheter of claim 40, further comprising a pull wire to move the plunger relative to the vent hole.

43. The catheter of claim 37, wherein the flow control device includes a plug having a plurality of holes to leak the fluid having the predetermined pressure.

44. The catheter of claim 37, wherein the flow control device includes a proximal tip portion, a distal tip portion movable relative to the proximal tip portion, and a spring between the proximal tip portion and the distal tip portion.

45. The catheter of claim 44, wherein the proximal tip portion is mounted on the catheter shaft, and wherein the balloon is coupled to the distal tip portion.

46. The catheter of claim wherein the distal tip portion is slidable on the proximal tip portion.

47. The catheter of claim 44, wherein the distal tip portion is rotatable on the proximal tip portion.

48. The catheter of claim 47, wherein the distal tip portion is threadably coupled to the proximal tip portion.

49. The catheter of claim 44, wherein the distal tip portion includes a vent hole, wherein the proximal tip portion includes a fluid port, and wherein the distal tip portion is movable relative to the proximal tip portion to align the vent hole with the fluid port.

50. The catheter of claim 37, wherein the catheter shaft has a shaft diameter less than or equal to 5 French.

51. The catheter of claim 37, wherein the flow control device is a check valve having an inlet port to receive fluid passing through the interior from the fluid lumen, wherein the check valve is configured to vent the fluid to a surrounding environment when the fluid has a predetermined pressure.

52. The catheter of claim 51, wherein the check valve includes a sealing plunger backing an elastomeric valve, and wherein the elastomeric valve has a crack pressure equal to the predetermined pressure.

53. A catheter, comprising:
a catheter shaft having a fluid lumen;
a balloon mounted on the catheter shaft and having an interior in fluid communication with the fluid lumen;
an ultrasound transducer in the interior; and
a flow control device having an inlet port to receive fluid passing through the interior from the fluid lumen, wherein the flow control device includes a plug having a plurality of holes to leak the fluid to a surrounding environment when the fluid has a predetermined pressure.

54. A catheter, comprising:
a catheter shaft having a fluid lumen;
a balloon mounted on the catheter shaft and having an interior in fluid communication with the fluid lumen;
an ultrasound transducer in the interior; and
a flow control device having an inlet port to receive fluid passing through the interior from the fluid lumen, wherein the flow control device includes a proximal tip portion, a distal tip portion movable relative to the proximal tip portion, and a spring between the proximal tip portion and the distal tip portion, and wherein the flow control device is configured to vent the fluid to a surrounding environment when the fluid has a predetermined pressure.

55. The catheter of claim 54, wherein the distal tip portion is slidable on the proximal tip portion.

56. The catheter of claim 54, wherein the distal tip portion is rotatable on the proximal tip portion.

57. The catheter of claim 56, wherein the distal tip portion is threadably coupled to the proximal tip portion.

58. The catheter of claim 54, wherein the distal tip portion includes a vent hole, wherein the proximal tip portion includes a fluid port, and wherein the distal tip portion is movable relative to the proximal tip portion to align the vent hole with the fluid port.

59. A catheter, comprising:
a balloon having an interior;
an ultrasound transducer in the interior; and
a catheter shaft including an outer member having a central lumen, an inner member extending through the central lumen and having a fluid lumen to deliver fluid to the interior, and one or more steering wires extending through the central lumen between an inner wall of the outer member and an outer wall of the inner member, wherein the one or more steering wires connect to the outer member or the inner member at an anchor point proximal to the balloon.

60. The catheter of claim 59, wherein the one or more steering wires includes a first steering wire diametrically opposite a second steering wire.

61. The catheter of claim 60, wherein one or more of the first steering wire or the second steering wire includes an electrical cable to deliver energy to the ultrasound transducer.

62. The catheter of claim 59, wherein the catheter shaft has a plurality of shaft sections, each shaft section having a respective stiffness.

63. The catheter of claim 62, wherein the plurality of shaft sections includes a distal section, an intermediate section, and a proximal section, wherein the respective stiffness of the distal section is less than the respective stiffness of the intermediate section, and wherein the respective stiffness of the intermediate section is less than the respective stiffness of the proximal section.

64. The catheter of claim 63, wherein the inner member has a wall, and wherein the wall is thinner at the distal section than at the intermediate section.

65. The catheter of claim 59, wherein the flow control device includes at least one of a check valve;
a vent hole extending through an outer wall from an internal channel, and a plunger movable within the internal channel relative to the vent hole;
a plug having a plurality of holes to leak the fluid having the predetermined pressure; and
a proximal tip portion, a distal tip portion movable relative to the proximal tip portion, and a spring between the proximal tip portion and the distal tip portion.
